(19) United States
(12) Patent Application Publication
(10) Pub. No.: US 2025/0128498 A1
(43) Pub. Date: Apr. 24, 2025

(54) NONWOVEN AGGREGATE AND METHOD OF MAKING A NONWOVEN AGGREGATE

(71) Applicants: OPTIMUM TECHNOLOGY IP LLC, Douglas (GB); REIFENHÄUSER GMBH & CO. KG MASCHINENFABRIK, Troisdorf (DE)

(72) Inventors: Patrick Bohl, Hennef (DE); Michael MASS, Roesrath (DE); Kim Patchett, Grays, Essex (GB); Sebastian SOMMER, Troisdorf (DE); Tobias WAGNER, KOELN (DE)

(21) Appl. No.: 18/727,554

(22) PCT Filed: Jan. 20, 2023
(86) PCT No.: PCT/EP2023/051441

§ 371 Date: Jul. 9, 2024

(30) Foreign Application Priority Data
Jan. 28, 2022 (DE) 10 2022 102 084.3

(51) Int. Cl. B32B 5/26; B32B 5/02; B32B 7/02; B32B 37/24; B32B 38/08; D04H 3/018; D04H 3/147; D04H 3/16

(52) U.S. Cl. CPC B32B 5/267; B32B 5/022; B32B 7/02; B32B 37/24; B32B 38/08; D04H 3/018; D04H 3/147; D04H 3/16; B32B 2250/20; B32B 2260/021; B32B 2262/124; B32B 2262/16; B32B 2305/026; B32B 2305/20; B32B 2305/30; B32B 2307/726; B32B 2307/737; D10B 2401/10

(57) ABSTRACT

The invention relates to a nonwoven unit comprising at least one nonwoven layer having at least one first layer and at least one second layer. The second layer is in the form of a nonwoven layer composed of continuous filaments. The average pore size of pores formed in the first layer is smaller than the average pore size of the pores between the filaments of the second layer. The second layer has functional particles which are embedded in the pores between the filaments of the second layer. The first layer forms a barrier preventing the penetration of particles or functional particles having a size greater than 150 μm.

The invention relates to a nonwoven aggregate with at least one nonwoven lamina with at least one first layer and at least one second layer, where the second layer is a nonwoven layer of continuous filaments. The nonwoven aggregate has embedded functional particles. In addition, the invention relates to a method and an apparatus for making a nonwoven aggregate.

Nonwoven aggregates of the above-mentioned type are generally known in practice in various embodiments. Such nonwoven aggregates are used for example in hygiene products such as diapers, incontinence products and sanitary napkins or in air filters, in medical products and in protective equipment in the medical, biological or chemical field. The functional particles can for example have absorbent, adsorbent, antiseptic or antifungal properties. In such nonwoven aggregates with embedded functional particles, it is desirable that the embedded functional particles move as little as possible within the nonwoven aggregate. Furthermore, in the case of nonwoven aggregates with embedded functional particles, it is desirable that a particle-size gradient results with regard to the distribution of the embedded functional particles across the thickness of the nonwoven aggregate, i.e. transversely, in particular perpendicular or essentially perpendicular to its planar extension. If such a particle-size gradient is present after the functional particles have been embedded, it is undesirable for the functional particles to move or shift within the nonwoven aggregate. With the nonwoven aggregates known to date, the realization of a distribution of the functional particles with a particle-size gradient and the simultaneous prevention of particle movement or displacement after incorporation of the functional particles into the nonwoven aggregate is not satisfactory and/or not economically feasible. In addition, it has been shown that the known measures generally only allow a very coarse gradation of the size distribution of embedded functional particles across the thickness of the nonwoven aggregates. In this respect, the desired nonwoven aggregate properties of the known nonwoven aggregates can only be set or adjusted with little flexibility and finesse. A satisfactory compromise between the economical production of the nonwoven aggregates on the one hand and the simple and functionally reliable realization of a gradient of the particle size across the thickness of the nonwoven aggregate in combination with at least extensive avoidance of movement or displacement of the embedded functional particles on the other hand has not yet been achieved. Although the fixation of functional particles in nonwoven aggregates by binders or adhesives is known, this is complex and can influence the properties of the embedded functional particles in an undesirable way. In addition, the binders or adhesion agents can cause skin irritation or allergies in the event of skin contact. In this respect, there is a need for improvement. This is where the invention comes in.

In contrast, the object of the invention is to provide a nonwoven aggregate of the above-mentioned type in which a gradient of the particle size distribution can be realized simply and reliably, at least in certain areas or sections across the thickness of the nonwoven aggregate, and in which movement or displacement of the functional particles within the nonwoven aggregate can be largely avoided after the functional particles have been put into the nonwoven aggregate. In addition, the invention has the further object of developing a method of making such a nonwoven aggregate and an apparatus for making such a nonwoven aggregate.

To attain these objects, the invention teaches a nonwoven aggregate with at least one nonwoven lamina with at least one first layer and at least one second layer, the second layer being in the form of a nonwoven layer of continuous filaments, wherein
the average pore size of pores formed in the first layer is smaller than the average pore size of the pores between the filaments of the second layer,
the second layer comprises functional particles that are embedded in the pores between the filaments of the second layer and
the first layer forms a barrier against penetration by particles or functional particles with a size greater than 150 μm, preferably greater than 100 μm, preferably greater than 50 μm, very preferably greater than 25 μm, particularly preferably greater than 10 μm and very particularly preferably greater than 0.5 μm.

When the term “layers” of filaments or continuous filaments or a nonwoven lamina with layers of filaments or continuous filaments is used here or in the following, this means that the layers of filaments or continuous filaments are spun from only one and the same spinning beam. When the term “laminae” or “nonwoven laminae” of filaments or continuous filaments is used here or below, this means that the individual laminae are each made with a separate spinning beam. “Endless” filaments differ from staple fibers, which have significantly shorter lengths of 10 mm to 60 mm for example, as opposed to their virtually endless length.

According to the invention, the first layer forms a barrier against penetration by particles or functional particles with a size greater than 150 μm, preferably greater than 100 μm, preferably greater than 50 μm, very preferably greater than 25 μm, particularly preferably greater than 10 μm and very particularly preferably greater than 0.5 μm. This is achieved in particular by the average pore size of pores formed in the first layer being smaller than the average pore size of the pores between the filaments of the second layer, and/or by the consolidation or final consolidation of the nonwoven aggregate. The mean pore size is an equivalent for the density of the nonwoven structure and/or the barrier resistance to the penetration of particles of the specified size or size distribution. The fact that a layer or a lamina forms a barrier against the penetration of particles or functional particles means in particular that this layer or lamina prevents or substantially prevents the passage of particles of the specified size. Moreover, it is within the scope of the invention that the nonwoven aggregate according to the invention is a bonded or final-bonded nonwoven aggregate or a nonwoven laminate.

In the context of the invention, the term average pore size refers in particular to the average pore size or the average pore volume of the pores. In the context of the invention, the average pore size or the ratio of the average pore size of two layers, of two layers or of one layer and one layer according to one embodiment can be determined by porometry using a porosimeter or an autoporosimeter. For this purpose, the nonwoven aggregate is preferably separated into its individual layers and/or laminae and the pore-size distribution of the pores of the individual layers and/or laminae is determined by porometry. Reference is made to WO 2011/053677 [U.S. Pat. No. 9,714,484] and in particular to pages 18 to 20. Additionally or alternatively, the mean pore size or the ratio of the mean pore size of two layers, two laminae or one layer and one lamina can preferably be determined by microcomputed tomography (μCT) by three-dimensional scanning. As with conventional X-ray computed tomography, microcomputed tomography enables the nondestructive testing of materials and components with regard to local differences in density. This allows pores, blowholes and cracks as well as different phases and component parts to be detected and analyzed. For this purpose, a virtual 3D model of the material or component is remade from radiographs at different angles using 3D reconstruction. In contrast to medical tomography, the resolution can be in the micrometer range depending on the sample or component size. Microcomputed tomography is particularly suitable for examining fibrous materials, their structure and gradients. For this purpose, a preferably cubic sample with an edge length of 8 mm for example is taken from the nonwoven aggregate and scanned with a resolution of 4 μm voxel size, for example. A digital twin of the material sample can be calculated from the raw scan data obtained. Reference is made to WO 2020/103964 [US 2022/0008263] and in particular to pages 49 to 53. The structural model or digital twin can preferably be generated using the FiberFind module from Math2Market GmbH. The pores or cavities between the fibers can be measured on a structural model or digital twin generated in this way. Preferably, modules such as MathDict and/or PoroDict from Math2Market GmbH can be used for this purpose for analyzing properties of porous media. In this way, the average pore size or the ratio of the average pore sizes of the layers or laminae in the nonwoven aggregate according to the invention can be determined. In addition or as an alternative to determining the pore size, a gray value analysis of sections through a μCT model can be carried out for the relevant layers within the scope of the invention in order to determine the density of the nonwoven structure in a simplified manner. For this purpose, a certain number of sections are placed across the thickness, for example five or ten sections, the grey values of each section are binarized in black and white (0/255) using a threshold value method and then the mean grey value of the layers is determined. Finally, the mean gray values of the different slices can be compared. The higher (brighter) the gray value, the denser the fleece structure.

A particularly preferred embodiment of the nonwoven aggregate according to the invention is characterized in that at least one third layer applied, in particular directly applied, to the second layer is present in the form of a nonwoven layer of continuous filaments or at least one nonwoven second lamina of continuous filaments applied, in particular directly applied, to the second layer is present, this third layer or the nonwoven second lamina preferably having an average pore size of the pores between the filaments that is smaller than the average pore size of the pores between the filaments of the second layer. Preferably, the third layer or the nonwoven second lamina forms a barrier against the penetration of particles or functional particles with a size greater than 150 μm, preferably greater than 100 μm, preferably greater than 50 μm, very preferably greater than 25 μm, particularly preferably greater than 10 μm and very particularly preferably greater than 0.5 μm. In the nonwoven aggregate according to the invention, it is possible that an intermediate layer of continuous filaments or an intermediate layer of continuous filaments is arranged or applied between the second layer and the third layer or the nonwoven second lamina. According to one embodiment, the intermediate layer or intermediate layer can have an average pore size of the pores between the filaments that is larger than the average pore size of the pores between the filaments of the second layer. With regard to the third layer, it is also possible that this third layer is made by partial consolidation, in particular by thermal partial consolidation, of the outer face of the second layer directed toward the outside of the nonwoven aggregate.

It is within the scope of the invention that the mean pore size of pores formed in the first layer is smaller than the mean pore size of the pores between the filaments of the second layer and/or that the third layer or the nonwoven second lamina has a mean pore size of the pores between the filaments that is smaller than the mean pore size of the pores between the filaments of the second layer. Expediently, the average pore size of the first layer corresponds to a maximum of 50%, preferably a maximum of 25%, preferably a maximum of 15% of the average pore size of the second layer.

According to a preferred embodiment of the invention, no functional particles or essentially no functional particles are embedded in the pores of the third layer or in the pores of the nonwoven second lamina. It is possible that functional particles are embedded in the first layer or in the pores of the first layer. Such functional particles embedded in the pores of the first layer are expediently functional particles whose average size is smaller than the average size of the functional particles embedded in the pores of the second layer. According to the embodiment in which functional particles are also embedded in the pores of the first layer, the first layer thus acts both as a barrier and as a layer for embedding functional particles of small size.

It has been proven effective when the third layer or the nonwoven second lamina is an outwardly closing layer or lamina of the nonwoven aggregate. According to another preferred embodiment, the third layer or the nonwoven second lamina is followed by a further top layer of continuous filaments or a top layer of continuous filaments. It is also preferred that the first layer of the nonwoven aggregate is an outwardly closing layer of the nonwoven aggregate. In this way, the nonwoven aggregate according to the invention is preferably terminated by the first layer in the direction of a first nonwoven aggregate outer face or lower face and terminated by the third layer or the nonwoven second lamina in the direction of a second nonwoven aggregate outer face or upper face, in particular opposite the first nonwoven aggregate outer face. Alternatively, the nonwoven aggregate according to the invention is preferably terminated by the first layer in the direction of a first nonwoven aggregate outer face or lower face and terminated by a top layer of continuous filaments or a top layer of continuous filaments in the direction of the second nonwoven aggregate outer or top face.

A particularly preferred embodiment of the nonwoven aggregate according to the invention is characterized in that the functional particles are embedded in the pores of the second layer without adhesives or binders. In the context of the invention, the fact that the functional particles are embedded in the pores of the second layer without adhesives or binders means in particular that there is no binder or adhesive provided for fixing the functional particles in the pores of the second layer. In particular, no adhesive or hot-melt adhesive for fixing the functional particles in the pores of the second layer is present in the nonwoven aggregate. In the context of the invention, a low-melting-point binder of multicomponent filaments, in particular of bicomponent filaments, is not regarded as an adhesion agent or binder or adhesive or hot-melt adhesive. In the context of the invention, the functional particles are preferably fixed in the pores of the second layer or in the nonwoven aggregate by the configuration of the layers of the aggregate and their properties, as well as the subsequent consolidation or final consolidation of the nonwoven aggregate. The embodiment, according to which the functional particles are embedded in the pores of the second layer without adhesives or binders, is based on the discovery that undesirable impairment of the functionality of the functional particles by adhesives or binders is avoided. In addition, skin irritations or allergies, which can sometimes result from skin contact with a binder or adhesion agent, can be avoided. It should also be understood that the entire nonwoven aggregate is preferably free of adhesives or binders and, in this respect, no adhesives or binders are present in the nonwoven aggregate for fixing the functional particles in the pores of the nonwoven aggregate.

It is within the scope of the invention that the functional particles are at least in part absorption particles, in particular for absorption of fluid media and/or at least in part adsorption particles. The fluid media may expediently be water-based fluid media. According to one embodiment, a fluid medium may be urine. It is also within the scope of the invention that the functional particles are at least in part adsorption particles, in particular for the adsorption of gases and/or solutions and/or liquids at the molecular level. According to one embodiment of the invention, the functional particles are organic and/or inorganic functional particles. Advantageously, the functional particles are formed on the basis of at least one component selected from the group: “polyacrylate, in particular sodium polyacrylate, silver salt, in particular silver chromate, sulfonated pentablock copolymer, activated carbon, copper salt, in particular copper sulfide, elemental iodine.” According to a preferred embodiment, the functional particles comprise at least one component selected from the above-mentioned group and in particular sodium polyacrylate or essentially sodium polyacrylate. In the context of the invention, the functional particles may have at least one property selected from the group: “absorption capacity, adsorption capacity, antiseptic properties, microbiocidal properties, antifungal properties, drying properties, hydrophilic, hydrophobic, metallic, magnetic, conductive, nonconductive.” It is also possible for the functional particles to have a combination of several of these properties.

According to a preferred embodiment of the invention, the functional particles have an average particle size of from 10 μm to 3000 μm, preferably from 20 μm to 2500 μm, particularly preferably from 40 μm to 2000 μm, most preferably from 50 μm to 1250 μm and most preferably from 100 μm to 1000 μm.

It is possible that the first layer of the nonwoven aggregate according to the invention is a film, in particular a plastic film. According to a further preferred embodiment, the first layer can be a separate base nonwoven layer made with a separate spinning beam. The base nonwoven layer can be formed from continuous filaments. It is possible that the base nonwoven layer is a spunbond layer and/or a meltblown layer. In principle, the first layer can also be an aggregate of at least one film or plastic film and a base nonwoven layer of filaments or continuous filaments, whereby the base nonwoven layer can be a meltblown layer and/or a spunbond layer.

According to a particularly preferred embodiment of the nonwoven aggregate according to the invention, the first layer is a nonwoven layer of continuous filaments. Preferably, the first layer and/or the second layer and/or the third layer is a spunbond nonwoven layer and/or the nonwoven second lamina is a spunbond nonwoven lamina. In the context of the invention, however, a nonwoven layer of continuous filaments or a nonwoven layer of continuous filaments can also be a meltblown nonwoven layer or a meltblown nonwoven layer. Preferably, the first layer and/or the second layer and/or the third layer is a spunbond nonwoven layer and/or the nonwoven second lamina is a spunbond nonwoven layer. It has been proven effective when the first layer and the second layer and the third layer or the nonwoven second lamina are a spunbond nonwoven layer or a spunbond nonwoven lamina.

It is particularly preferred that the first nonwoven lamina or the first layer and the second layer are spun from only one spinning beam or from only one spinneret. In this case, the first layer and the second layer are a nonwoven layer made of continuous filaments and the first nonwoven lamina is a nonwoven layer made of continuous filaments. If two layers are spun from only one spinning beam or from only one spinneret, it is preferred that the filaments are spun from this one spinning beam or from this one spinneret in such a way that the properties of the deposited filaments differ in such a way that two nonwoven layers of continuous filaments with different properties result or are deposited on top of each other. This can be achieved by varying the properties of the spun filaments in the machine direction (MD direction) and/or transverse to the machine direction (CD direction), preferably only in the machine direction (MD direction), along the spinning beam or the spinneret. This is explained in more detail below. In this context, it is preferable that the properties of the filaments spun from only one spinning beam or from only one spinneret are the same or essentially the same in the direction transverse to the machine direction (CD direction), so that the individual deposited layers are homogeneous or essentially homogeneous throughout their entire areas with regard to these filament properties. The fact that two nonwoven layers of continuous filaments are spun from only one spinning beam or only one spinneret can be recognized in the resulting nonwoven aggregate in particular by a short transition area across the thickness of the nonwoven aggregate, so that there is preferably no sharp transition between the two nonwoven layers, as would be the case with two nonwoven laminae of continuous filaments spun from two separate spinning beams. This can also be shown for example by delamination tests of the layers or laminae of a nonwoven aggregate and/or by microcomputed tomography (see above).

A preferred embodiment of the invention is characterized in that the third layer is spun from the same spinning beam or from the same spinneret as the first and second layers. In this case, the third layer is expediently a nonwoven layer of continuous filaments. It is possible that the third layer is made by partial consolidation, in particular by thermal partial consolidation, of the outer face of the second layer directed toward the outside of the nonwoven aggregate. According to an alternative embodiment, the nonwoven second lamina is spun from a further or second spinning beam or from a further or second spinneret. It is also possible that the nonwoven second lamina is applied to the second layer by an unwinder as a separate nonwoven layer.

According to the invention, the second layer is a nonwoven layer of continuous filaments. Preferably, the first layer is also a nonwoven layer of continuous filaments. Preferably, the nonwoven aggregate has a third layer that is a nonwoven layer of continuous filaments or the nonwoven aggregate has a nonwoven second lamina of continuous filaments. In this context, it has proven useful for the filaments of the first layer and/or the filaments of the third layer or the nonwoven second lamina to have a lower average titer than the filaments of the second layer. Further preferably, the filaments of the third layer or the nonwoven second lamina have a higher average titer than the filaments of the first layer. According to a particularly preferred embodiment of the nonwoven aggregate according to the invention, the average titer of the filaments of the first layer is 0.1 den to 5 den, preferably 0.2 den to 2.5 den, preferably 0.3 den to 2.0 den, particularly preferably 0.4 den to 1.5 den, most preferably 0.5 den to 1.3 den, for example 1.1 den or about 1.1 den. It has been proven effective when the average titer of the filaments of the second layer is 2.0 den to 12 den, preferably 3.0 den to 10 den, preferably 4.0 den to 8.0 den and particularly preferably 5.5 den to 6.5 den, for example 6.0 den or about 6.0 den. With regard to the third layer or the nonwoven second lamina, it is preferred that the average titer of the filaments used is 0.1 den to 5.0 den, preferably 0.2 den to 2.5 den, preferably 0.3 den to 2.0 den, particularly preferably 0.4 den to 1.5 den, most preferably 0.5 den to 1.3 den, for example 1.1 den or about 1.1 den. According to one embodiment, the specified den ranges result in particular when using polypropylene/polyethylene as filament components of bicomponent filaments. Filament titers in μm are then preferably obtained by converting the specified den values assuming a density of 0.92 g/cm³.

The embodiment of the nonwoven aggregate according to the invention in which the filaments of the first layer and/or the filaments of the third layer or the nonwoven second lamina have a lower average titer than the filaments of the second layer and where preferably the filaments of the third layer or the nonwoven second lamina have a higher average titer than the filaments of the first layer, is based on the discovery that the different titers of the individual layers or laminae allow the different pore sizes of the individual layers or laminae to be adjusted in a functionally reliable manner. In addition, it is preferably ensured that the third layer or the nonwoven second lamina initially enables injecting functional particles through this layer into the nonwoven aggregate or into the second layer and then, after the consolidation or final consolidation of the nonwoven aggregate, acts in particular as a barrier, as already described above.

According to a highly recommended embodiment of the invention, the filaments of the first layer and/or the filaments of the second layer and/or the filaments of the third layer or the nonwoven second lamina are multicomponent filaments, in particular bicomponent filaments. Preferably, the multicomponent filaments have at least one low-melting-point binder and particularly preferably the multicomponent filaments are crimped continuous filaments. It has proven to be the case that the filaments of the first layer and the filaments of the second layer and the filaments of the third layer or the nonwoven second lamina are multicomponent filaments, in particular bicomponent filaments, and that the multicomponent filaments preferably have at least one low-melting-point binder and are particularly preferably crimped continuous filaments.

In this context, it has also proven to be the case that the degree of crimp of the filaments of the first layer and/or the filaments of the third layer or the second layer is lower than the degree of crimp of the filaments of the second layer. In the context of the invention, the fact that the degree of crimp of the filaments of one layer or lamina is lower than the degree of crimp of the filaments of another layer or lamina means in particular that the filaments have fewer crimps or bends per centimeter of filament length (number of bends) and/or a larger crimp diameter or bend diameter than the filaments of the other layer or lamina. The number of crimp loops or crimp arcs per centimeter of filament length is measured in particular according to Japanese standard JIS L10151518 by counting the crimps under a bias of 2 mg/den in ( 1/10 mm), based on the stretched length of the filaments. A sensitivity of 0.05 mm is used to determine the number of crimps. The measurement is conveniently carried out with a “Favimat” apparatus from TexTechno, Germany. Please refer to the publication “Automatic Crimp Measurement on Staple Fibers”, Denkendorf Kolloquium, “Textile Mess und Prüftechnik”, 9.11.99, Dr. Ulrich Mörschel (in particular page 4, FIG. 4). The filaments (or the filament sample) are removed as a filament ball from the deposit or from the deposition belt before further consolidation and the filaments are separated and measured. The sheet diameter is conveniently measured by placing the nonwoven fabric to be measured under a microscope and generating a still image using suitable magnification, in which the sheet diameter can be measured. In the case of nonwoven aggregates or nonwoven laminates with several layers, the optical system must be focused on the surface of each visible layer so that the other surfaces or layers lie as far as possible outside the depth of field. Due to the random distribution of the filaments or the random distribution of the sheet diameters, at least 25 measurements are required in each case. The arithmetic mean value is specified. This embodiment is based on the discovery that different pore sizes of the layers or laminae can preferably be set due to the different degree of crimp of the layers or laminae. According to one embodiment, the first layer or the first nonwoven layer of continuous filaments has a number of sheets in the range 0 to 3 per cm and the second layer has a number of sheets in the range 2 to 6 per cm, with the second layer preferably having a greater number of sheets than the first layer. For a more open structure, larger crimp sheet amplitudes are particularly preferred.

It has already been explained above that the filaments of the first layer and/or the filaments of the second layer and/or the filaments of the third layer or the nonwoven second lamina are multicomponent filaments, in particular bicomponent filaments, and that the multicomponent filaments expediently have at least one low-melting-point binder. It is within the scope of the invention that the proportion of the low-melting-point binder in the multicomponent filaments of the first layer and/or the third layer or nonwoven second lamina is higher than the proportion of the low-melting-point binder in the multicomponent filaments of the second layer. Expediently, the proportion of the low-melting-point binder component in the multicomponent filaments of the first layer is 10 to 70 wt %, preferably 15 to 65 wt %, preferably 20 to 60 wt %, particularly preferably 35 to 55 wt %, for example 50 wt % or about 50 wt % based on the components of the multicomponent filaments of the first layer. Further preferably, the proportion of the low-melting-point binder component in the multicomponent filaments of the second layer is 5 to 55 wt %, preferably 10 to 30 wt %, particularly preferably 15 to 30 wt %, especially preferably 15 to 25 wt %, for example 20 wt % or about 20 wt % based on the components of the multicomponent filaments of the second layer. With regard to the third layer or the nonwoven second lamina, it is preferred that the proportion of the low-melting-point binder in the multicomponent filaments is 15 wt % to 60 wt %, preferably 20 wt % to 50 wt %, preferably 22 wt % to 40 wt %, for example 30 wt % or about 30 wt %, based on the components of the multicomponent filaments of the third layer or the nonwoven second lamina.

The following information regarding the components of the multicomponent filaments preferably applies to all layers or laminae of the nonwoven aggregate of multicomponent filaments, in particular of bicomponent filaments: It is particularly preferred that the at least one low-melting-point binder of the multicomponent filaments consists or essentially consists of polyethylene. If “essentially consists” is stated here or below in relation to the components of the multicomponent filaments or the bicomponent filaments and in particular to a plastic or to a polymer, this means in particular that the component or the polymer is present to at least 95 wt %, preferably to at least 97 wt % and preferably to at least 98 wt %. The remaining percent by weight can be formed in particular by additives such as plasticizers, fillers, dyes and the like. It is further preferred that the at least one further component or the second component of the multicomponent filaments, in particular the bicomponent filaments, consists or essentially consists of polypropylene or of a polyester. By the second or further component is meant in particular the second component present in addition to the low-melting-point binder or a further component present in addition to the low-melting-point binder. The second or further component of the multicomponent filaments or bicomponent filaments then forms the high-melting-point component in particular. For the further or second component, at least one polypropylene copolymer can be used instead of polypropylene or in addition to polypropylene and at least one polyester copolymer can be used instead of the polyester or in addition to the polyester. Polyethylene terephthalate (PET) and/or polylactide (PLA) is particularly suitable as a polyester and PET copolymer (CoPET) is particularly suitable as a polyester copolymer. A recommended embodiment of the invention is characterized in that the at least one further component or the second component of the multicomponent filaments or bicomponent filaments consists or essentially consists of at least one plastic from the group “polypropylene, polypropylene copolymer, polyethylene terephthalate (PET), polylactide (PLA), PET copolymer (CoPET).” According to a particularly preferred embodiment of the invention, the low-melting-point binder of the multicomponent filaments or bicomponent filaments consists of polyethylene or essentially of polyethylene and the second or further component of the multicomponent filaments or bicomponent filaments, in particular the high-melting-point component, consists of polypropylene or essentially of polypropylene.

If, according to a preferred embodiment, the filaments of the second layer are multicomponent filaments, in particular bicomponent filaments, the multicomponent filaments preferably having at least one low-melting-point binder, it has proved particularly useful for the binder content of the multicomponent filaments of the second layer to be dimensioned such that in the event of swelling of the functional particles, in particular in the event of swelling of absorption particles as a result of absorption of fluid media, at least partial breaking of the bonds between the filaments of the second layer is possible, and where for this purpose the binder content of the multicomponent filaments is 5 to 55 wt %, preferably 10 to 30 wt %. wt %, preferably 10 to 30 wt % and in particular 15 to 25 wt % based on the components of the multicomponent filaments. This embodiment is based on the discovery that a sufficiently low proportion of the low-melting-point binder in the multicomponent filaments or bicomponent filaments of the second layer and, in particular, due to the resulting relatively low bond strength when the functional particles, in particular the absorption particles, swell, at least partial breaking of the filament-filament connection points is possible, so that the functional particles do not emerge from the second layer or essentially do not emerge from the second layer during swelling.

Within the scope of the invention, it is possible in principle for the multicomponent filaments or bicomponent filaments of the layers or laminae of the nonwoven aggregate to be multicomponent filaments or bicomponent filaments with a core/sheath configuration, in particular with an eccentric core/sheath configuration, with a side-by-side configuration, with a multilobal configuration, with a trilobal configuration or similar cross-sectional configurations.

It is within the scope of the invention that the multicomponent filaments of the first layer and/or of the third layer or of the nonwoven second lamina are multicomponent filaments with a core/sheath configuration, in particular with an eccentric core/sheath configuration, and where according to a preferred embodiment the multicomponent filaments of the second layer are multicomponent filaments with a side-by-side configuration. It is possible that the multicomponent filaments of the first layer are multicomponent filaments with a centric core/sheath configuration. It is also possible that the multicomponent filaments of the third layer or the nonwoven second lamina are multicomponent filaments with side-by-side configuration. If the multicomponent filaments of the first layer are multicomponent filaments with an eccentric core/sheath configuration according to a preferred embodiment, it is within the scope of the invention that both the sheath of the filaments and the core of the filaments are circular as seen in the filament cross-section. According to a further preferred embodiment of the invention, the multicomponent filaments of the first layer are multicomponent filaments with an eccentric core/sheath configuration and the core of these filaments is designed in the shape of a circular segment as seen in the filament cross-section and has a circular arc-shaped circumferential section and a linear circumferential section with respect to its circumference, so that a D-shape of the core results as seen in the filament cross-section. If, according to a preferred embodiment of the invention, the multicomponent filaments of the first layer and the third layer or of the nonwoven second lamina are multicomponent filaments with an eccentric core/sheath configuration, it is also preferred that a spacing of the center of gravity of the core from the center of gravity of the sheath in the multicomponent filaments with an eccentric core/sheath configuration of the first layer is smaller than a spacing of the center of gravity of the core from the center of gravity of the sheath of the multicomponent filaments with an eccentric core/sheath configuration of the third layer or the nonwoven second lamina.

It is within the scope of the invention that the base weight of the nonwoven aggregate is 50 to 350 g/m², preferably 80 to 300 g/m², preferably 100 to 250 g/m², based on the aggregate comprising the first layer, second layer and third layer or nonwoven second lamina. The base weight refers in particular to the aggregate without functional particles or before the functional particles are applied and incorporated. Conveniently, the weight of the functional particles embedded in the nonwoven aggregate is 200 g/m² to 600 g/m², preferably 300 g/m² to 500 g/m² relative to the surface area of the nonwoven aggregate. It has been proven effective when the ratio of the weight of the functional particles embedded in the nonwoven aggregate in relation to the surface area of the nonwoven aggregate to the base weight of the nonwoven aggregate is 2:1 to 50:1, preferably 3:1 to 30:1, preferably 3:1 to 20:1, particularly preferably 3:1 to 5:1. Further preferably, the average titer of the filaments of the aggregate of first layer, second layer and third layer or nonwoven second lamina according to a preferred embodiment of the invention is greater than 2 den, particularly preferably greater than 3 den.

According to a further preferred embodiment of the nonwoven aggregate according to the invention, the filaments of the first layer and the filaments of the second layer and the filaments of the third layer or of the nonwoven second lamina are multicomponent filaments, in particular bicomponent filaments, where the multicomponent filaments have at least one low-melting-point binder and where the average proportion of the low-melting-point binder relative to the components of the multicomponent filaments of the aggregate of first layer, second layer and third layer or nonwoven second lamina is less than 40 wt %, preferably less than 33 wt %. In this context, it is within the scope of the invention that the low-melting-point binder consists or essentially consists of polyethylene. It has been found that the binder content of the multicomponent filaments of the first layer is greater, preferably by at least 5%, than the average binder content of the multicomponent filaments of the first layer, the second layer and the third layer or the nonwoven second lamina.

It is preferred that the mass fraction of the first layer is 5 to 60 wt %, in particular 20 to 50 wt %, preferably 30 to 45 wt %, more preferably the mass fraction of the second layer is 30 to 85 wt %, in particular 35 to 80 wt %, preferably 40 to 75 wt % and further preferably the mass fraction of the third layer or the nonwoven second lamina is 4 to 15 wt %, in particular 5 to 14 wt %, preferably 6 to 13 wt % relative to the aggregate of first layer, second layer and third layer or nonwoven second lamina. In the context of the invention, the stated mass fractions are in particular the mass fractions without functional particles or before the application and incorporation of the functional particles. Preferably, the mass fraction of the second layer is greater than the mass fraction of the first layer and the third layer and further preferably, the mass fraction of the first layer is greater than the mass fraction of the third layer.

A particularly preferred embodiment of the nonwoven aggregate according to the invention is characterized in that the second layer has at least two, preferably two, partial layers. A first partial layer is preferably adjacent the first layer and a second partial layer is preferably adjacent the third layer or the nonwoven second lamina. According to a particularly preferred embodiment, the filaments of the first partial layer of the second layer preferably adjacent the first layer, have a lower average titer than the filaments of the second partial layer of the second layer preferably adjacent the third layer or the nonwoven second lamina, and/or where both partial layers contain multicomponent filaments, where the multicomponent filaments of the first partial layer have a higher binder content than the multicomponent filaments of the second partial layer.

It has been proven effective when the average titer of the filaments of the first partial layer 2 is 2 den to 6 den, preferably 2.5 den to 5.5 den, preferably 3 den to 5 den. It is further preferred that the average titer of the filaments of the second partial layer is 2 den to 9 den, preferably 3 den to 8 den, preferably 4 den to 7 den, for example 6 den or about 6 den. According to a preferred embodiment, the filaments of the first partial layer and/or the second partial layer are multicomponent filaments, in particular bicomponent filaments. Particularly preferred are the filaments of the first partial layer and/or the second partial layer multicomponent filaments with side-by-side configuration. It has been proven effective when the multicomponent filaments of the first partial layer and/or the second partial layer have at least one low-melting-point binder and that this low-melting-point binder consists or essentially consists in particular of polyethylene. Expediently, the binder content of the multicomponent filaments of the first partial layer is 15 to 40 wt %, preferably 20 to 35 wt %, for example 25 wt % or about 25 wt % relative to the components of the multicomponent filaments of the first partial layer. Further preferably, the binder content of the multicomponent filaments of the second partial layer is 5 to 25 wt %, preferably 10 to 20 wt %, for example 15 wt % or about 15 wt % based on the components of the multicomponent filaments of the second partial layer. Expediently, the further component or the second component of the multicomponent filaments, in particular of the bicomponent filaments, of the first partial layer and/or of the second partial layer consists of polypropylene or essentially of polypropylene.

It is also preferred that the mass fraction of the first partial layer is 15 to 30 wt %, in particular 20 to 30 wt %, and the mass fraction of the second partial layer is 40 to 70 wt %, in particular 50 to 60 wt %, based on the aggregate of first layer, second layer or first and second partial layer of the second layer, and third layer or nonwoven second lamina. The mass fractions here preferably refer to the aggregate without incorporated functional particles or before the application and incorporation of the functional particles. It is within the scope of the invention that the average pore size of the pores formed between the filaments of the first partial layer of the second layer is smaller than the average pore size of the pores formed between the filaments of the second partial layer of the second layer. Conveniently, the first partial layer of the first layer and the second partial layer of the third layer or the nonwoven second lamina are adjacent. If the nonwoven aggregate according to an embodiment described above comprises an intermediate layer between the second layer and the third layer or the nonwoven second lamina, it is possible that the intermediate layer has the above-described properties of the second partial layer and that the second layer has the above-described properties of the first partial layer and then has no second partial layer. According to one embodiment, it is also possible that the second layer has no first and second partial layer and that instead the second layer has the properties of the first partial layer of the second layer described above, where a nonwoven second lamina applied to the second layer comprises two nonwoven layers of continuous filaments, where a first layer of the nonwoven second lamina that is adjacent the second layer has the properties of the second partial layer of the second layer described above and where the second layer of the nonwoven second lamina has the properties of the third layer or nonwoven second lamina described above.

It is within the scope of the invention that the nonwoven aggregate has a top layer of continuous filaments above the third layer or the nonwoven second lamina. Preferably, the top layer is a spunbond nonwoven layer and particularly preferred are the filaments of the top layer multicomponent filaments, especially bicomponent filaments. It has been proven effective when the multicomponent filaments of the top layer are multicomponent filaments with a core/sheath configuration, in particular with an eccentric core/sheath configuration. Preferably, the multicomponent filaments of the top layer have at least one low-melting-point binder component. Further preferably, the low-melting-point binder consists of polyethylene or essentially of polyethylene. The further or second component of the multicomponent filaments or bicomponent filaments of the top layer is expediently made of polypropylene or essentially of polypropylene. It has been proven effective when the binder content of the multicomponent filaments of the top layer is more than 30 wt %, preferably more than 40 wt %, relative to the components of the multicomponent filaments of the top layer. It is further preferred that the average titer of the multicomponent filaments of the top layer is less than 3 den.

It is preferred that the nonwoven aggregate according to the invention is a consolidated or final-consolidated nonwoven aggregate or nonwoven laminate. Preferably, the nonwoven aggregate is a thermally consolidated or final-consolidated nonwoven aggregate. It is within the scope of the invention that the consolidation or final consolidation, in particular the thermal consolidation or final consolidation, of the nonwoven aggregate is carried out with the aid of a consolidator, in particular with the aid of a calender. According to one embodiment, the consolidator can have a flow-through oven and/or a hot calender. It is advisable to feed the aggregate pre-bonded in the oven directly to the hot calender without significant cooling in order to utilize the product waste heat for effective hot calendering.

It is within the scope of the invention that the second layer and/or the third layer or the nonwoven second lamina have hydrophilic properties and, in particular, have more hydrophilic properties than the first layer. Advantageously, the third layer or the nonwoven second lamina has more hydrophilic properties than the second layer. It is possible for the hydrophilic properties of a layer or lamina to be adjusted by admixing a hydrophilic additive to the melt from which the filaments are spun and/or by applying a hydrophilizing agent, in particular a hydrophilic lubricant, to the layer or lamina after spinning. In the context of the invention, the fact that a hydrophilizing agent is or is applied to a lamina or layer means in particular that the hydrophilizing agent is applied to the filaments or the deposited filaments of the lamina or layer. By adding a hydrophilic additive to the melt from which the filaments are spun, the fibers and thus indirectly the lamina or layer are given more hydrophilic properties. Applying a hydrophilizing agent or hydrophilic lubricant to the layer after spinning gives the layer more hydrophilic properties, but the hydrophilic properties of the filament material are not changed.

Preferably, a hydrophilizing agent, in particular a hydrophilic lubricant, is applied to at least the second layer and/or the third layer or the nonwoven second lamina to adjust the hydrophilicity of this layer or lamina. The hydrophilizing agent or hydrophilic lubricant is in particular a liquid hydrophilizing agent that dries or is dried after application. In the context of the invention, the fact that one layer or lamina is more hydrophilic than another layer or lamina in terms of its properties preferably means that the layer or lamina has more hydrophilic properties with respect to an aqueous sodium-chloride solution, preferably a 0.9% aqueous sodium-chloride solution, than the other layer or lamina. In the context of the invention, 0.9% aqueous sodium-chloride solution means in particular a 0.9 wt % solution of sodium chloride in deionized or distilled water. The measurement or determination of the hydrophilic properties of a layer or lamina of the nonwoven aggregate can be determined by determining the surface tension of the surface or the outside of the layer or lamina with respect to a test liquid, in particular with respect to a 0.9% aqueous sodium-chloride solution, by determining the time until this test liquid has penetrated into the layer or lamina. For this purpose, the nonwoven aggregate may first be separated into its individual layers and/or laminae. Additionally or alternatively, the contact angle between a test liquid and the layer or lamina can be determined in order to ascertain whether one layer or lamina has more hydrophilic properties than another layer or lamina. Preferably, a 0.9% aqueous sodium-chloride solution can also be used as the test liquid. If, when using a test liquid, no time difference in the penetration of the test liquid can be determined when comparing two layers or laminae, the wetting tension of the layers or laminae is preferably determined in accordance with DIN ISO 8296 “Plastics films and sheets—Determination of wetting tension.”

To attain these objects, the invention further teaches a method of making a nonwoven aggregate, in particular a nonwoven aggregate as described above, where at least a first layer is deposited on a receiving surface, in particular on a mesh deposition belt, and where at least a second layer in the form of a nonwoven layer of continuous filaments is made by spinning out the continuous filaments from a spinning beam and is deposited on the first layer, whereby functional particles are applied to the aggregate and the functional particles are embedded between the filaments of the second layer and where the first layer is made such that it forms a barrier against penetration by particles or functional particles with a size greater than 150 μm, preferably greater than 100 μm, preferably greater than 50 μm, very preferably greater than 25 μm, particularly preferably greater than 10 μm and very particularly preferably greater than 0.5 μm.

A particularly preferred embodiment of the process according to the invention is characterized in that the first layer is also made as a nonwoven layer of continuous filaments and the filaments for the first layer and the filaments for the second layer are spun with the same spinning beam. It is further preferred that at least a third layer in the form of a nonwoven layer of continuous filaments or at least a nonwoven second lamina of continuous filaments is made and deposited on the second layer.

According to a particularly preferred embodiment of the method according to the invention, the filaments for the first layer and the filaments for the second layer and the filaments for the third layer are spun with the same spinning beam. Then at least one spinning-beam property or at least one filament property from the group: “per-hole throughput, cross-sectional geometry of the multicomponent filaments, binder proportion of multicomponent filaments” is varied in order to produce three layers different from each other, or to deposit them on top of each other. Conveniently, viewed in the MD direction, the inlet side of the spinning beam is provided for making the first layer and the middle area for making the second layer and the outlet side for making the third layer. The three areas of the spinning beam intended for making the first layer, the second layer and the third layer then produce the three layers with the properties of the filaments described above. In this way, the properties of the layers can be set very precisely and accurately, resulting in a nonwoven aggregate in which properties such as the average pore size can be set very precisely and in fine increments. The selection and combination of spinning-beam properties or filament properties within a spinning beam is, as it were, a toolbox for making nonwoven layers from continuous filaments with precisely and finely adjustable and tunable properties. It is alternatively possible within the framework of the method according to the invention that the third layer is made by partial consolidation, in particular by thermal partial consolidation, of the outer face of the second layer directed toward the outside of the nonwoven aggregate.

According to an alternative embodiment of the method according to the invention, a nonwoven second lamina of continuous filaments is deposited on the second layer and this nonwoven second lamina of continuous filaments is spun with a separate spinning beam. It is possible for the nonwoven second lamina to be unwound from an unwinder or a roll and placed on the second layer. It is also possible that, as part of the method according to the invention, a top layer of continuous filaments is spun using a further separate spinning beam and is deposited on the third layer or on the nonwoven second lamina or on the second layer. It is possible for the top layer to be unwound from an unwinder or a roll and placed on the third layer or the nonwoven second lamina or on the second layer. When the nonwoven second lamina is deposited, the first layer and the second layer are preferably compacted by the process air of a second spinning beam. In the process according to the invention, this preferably makes it possible to reduce the average pore size of the first and second layers during the deposition of the nonwoven second lamina.

According to a preferred embodiment of the invention, the functional particles are applied to the second layer and introduced into the second layer and then, expediently, the third layer or the nonwoven second lamina is applied or directly applied to the second layer. According to a further preferred embodiment of the method according to the invention, the functional particles are applied to the third layer or the nonwoven second lamina, the third layer or the nonwoven second lamina being made such that the functional particles penetrate the third layer or the nonwoven second lamina at least before the third layer or the nonwoven second lamina is consolidated and/or compacted and are embedded at least for the most part between the filaments of the second layer. It is within the scope of the invention that after the application of the functional particles, in particular after the application of the functional particles to the third layer or the nonwoven second lamina or to the second layer, the functional particles are introduced into the nonwoven aggregate. Injecting the functional particles into the nonwoven aggregate or into the second layer is expediently effected by mechanical energy and/or by an electric field and/or by acoustic pressure, for example by sound. Preferably, the functional particles are injected into the nonwoven aggregate or into the second layer by acoustic pressure in the range from 100 Hz to 25 kHz, preferably in the range from 10 kHz to 18 kHz, particularly preferably in the range from 14 kHz to 16 kHz. The amplitude of the acoustic pressure is also preferably between 5 μm and 5000 μm, preferably between 50 μm and 150 μm and particularly preferably between 80 μm and 110 μm.

It is within the scope of the method according to the invention that a hydrophilizing agent, in particular a hydrophilic lubricant, is applied to the nonwoven aggregate. According to one embodiment, the hydrophilizing agent or the hydrophilic lubricant can be applied before/and or after, in particular after, the application of the functional particles to the nonwoven aggregate. Conveniently, the hydrophilizing agent or hydrophilic lubricant is applied to the nonwoven aggregate by spraying. Preferably, this is a liquid hydrophilizing agent or a liquid hydrophilic lubricant. A drying step is also preferably carried out after the application or spray application of the hydrophilic lubricant. If, according to a preferred embodiment of the method according to the invention, a first layer and a second layer and a third layer are spun with the same spinning beam, the application of the hydrophilizing agent or the hydrophilic lubricant preferably takes place after the application of the functional particles to the third layer and further preferably after Injecting the functional particles into the aggregate or into the second layer. If, according to one embodiment, a nonwoven second lamina of continuous filaments is applied to the second layer, the hydrophilizing agent or the hydrophilic lubricant is preferably applied before and/or after, preferably after, the nonwoven second lamina is applied to the second layer. If, according to one embodiment, a nonwoven second lamina is applied to the second layer, it is also preferred that the application and injection of the functional particles takes place before and/or after, in particular before, the spinning out of the filaments by the second spinning beam or the deposition of the nonwoven second lamina on the second layer.

It is within the scope of the method according to the invention that, after Injecting the functional particles into the nonwoven aggregate or into the second layer, a consolidating of the surface or of the third layer or of the nonwoven second lamina and/or a consolidating of the entire aggregate comprising first layer, second layer and third layer or nonwoven second lamina takes place. The consolidating of the third layer or the nonwoven second lamina after Injecting the functional particles into the nonwoven aggregate or into the second layer preferably leads to a sealing of this layer, so that the functional particles can first be introduced into the nonwoven aggregate through this layer or lamina and this sealed layer or lamina then acts as a barrier for the functional particles.

It is also possible in the context of the method according to the invention that, after the production and deposition of the first layer and the second layer and the third layer or the nonwoven second lamina, a preconsolidation or compaction of the deposited aggregate takes place and that the application and injection of the functional particles and/or the application of the hydrophilic lubricant is then carried out. According to a further preferred embodiment, preconsolidation or compaction of the aggregate comprising the first layer and the second layer and the third layer or the nonwoven second lamina takes place after application of the hydrophilizing agent or the hydrophilic lubricant. This is particularly advantageous, as the process heat of the preconsolidation or compaction can dry the residual moisture of the hydrophilic lubricant without additional effort. It is also within the scope of the invention that no preconsolidation of the nonwoven aggregate takes place and that the entire nonwoven aggregate is only consolidated or finally compacted after the application and injection of the functional particles and their introduction into the nonwoven aggregate and after the application of the hydrophilic lubricant or the hydrophilic lubricant.

As part of the method according to the invention, it is possible for the aggregate comprising the first layer and the second layer and the third layer or the nonwoven second lamina to be consolidated with as little compaction as possible before the functional particles are applied, such that the nonwoven aggregate is transportable. After this consolidating of the nonwoven aggregate comprising the first layer and the second layer and the third layer or the nonwoven second lamina, the nonwoven aggregate can initially be laid down or wound up so that the application and incorporation of the functional particles into the nonwoven aggregate can take place in a separate “offline” step. In such an embodiment, the application of the at least one hydrophilizing agent or the hydrophilic lubricant can take place before or after the application and incorporation of the functional particles.

It is within the scope of the method according to the invention that the nonwoven aggregate is cut to size for later use. Sufficient immobilization of the functional particles is intended to ensure that the functional particles can be held or fixed in the nonwoven aggregate despite the cutting. This is expediently achieved by sealing and/or welding the cut surface. The sealing and/or welding can be achieved by thermal and/or thermomechanical consolidating and/or mechanical treatment, in particular by laser, fluid, hot stamping or ultrasound. Preferably, the sealing or welding takes place in one process step combined with the cutting. In the case of a laser cut, the process parameters can be selected so that the edge areas melt and fuse and welded cut surfaces are formed on both sides of the cut on the nonwoven aggregate. This can also be done with an appropriately designed ultrasonic sonotrode, for example in the form of a roller.

To attain these objects, the invention further teaches an apparatus for making a nonwoven aggregate, in particular for making a nonwoven aggregate as described above and/or made using a method described above, where a spinning beam is provided for spinning out continuous filaments and a receiving surface, in particular a mesh deposition belt, is provided for deposition of the filaments to form a nonwoven web, where filaments for at least a first layer or first nonwoven layer can be made with the spinning beam and can be deposited on the receiving surface, where filaments for at least a layer nonwoven layer can be made with the same spinning beam, which filaments can be deposited on the first layer or the first nonwoven layer, where the spinning beam or the spinneret openings of the spinning beam being set up such that
the filaments of the first layer have a lower mean titer than the filaments of the second layer
and/or
the filaments of the first and second layers are crimped multicomponent filaments and the filaments of the second layer have a higher degree of crimp than the filaments of the first layer
and/or
the filaments of the first and second layers are multicomponent filaments with a low-melting-point binder component and the binder component content of the filaments of the first layer is higher than the binder component content of the filaments of the second layer, and
at least one applying and/or injecting device for applying and/or injecting functional particles onto or into the unit is downstream of the spinning beam in the machine direction.

According to a preferred embodiment, the spinning beam can also be used to produce filaments for at least a third layer or third nonwoven layer, which filaments can be deposited on the second layer or second nonwoven layers. According to an alternative embodiment, the apparatus has a second spinning beam for spinning continuous filaments for a nonwoven second lamina of continuous filaments, and the nonwoven second lamina can be deposited on the second layer. It is possible that the apparatus according to the invention has an unwinder for applying a top layer to the third layer or the nonwoven second lamina. The unwinder for applying the top layer is expediently downstream of the first spinning beam or the second spinning beam. If the apparatus has an unwinder for a top layer, it is preferred that the apparatus has an oven for thermally consolidating the top layer to the third layer or the nonwoven second lamina. It is further preferred that the apparatus according to the invention has at least one applicator for applying at least one hydrophilizing agent, in particular a hydrophilic lubricant, to the aggregate. The applicator is preferably connected downstream of the spinning beam in the machine direction and further preferably downstream of the applying and/or injecting device in the machine direction. According to a preferred embodiment, the applicator is a spray system, for example as a spray nozzle. The applicator or the spray system is then provided in particular for spray application of the at least one hydrophilizing agent or the hydrophilic lubricant. It is also possible that the applicator is a roller. In this case, the applicator or the application roller is intended in particular for the roller application of the at least one hydrophilizing agent or the hydrophilic lubricant (kiss-roll application).

The invention is based on the discovery that functional particles can be fixed very securely in the nonwoven aggregate according to the invention and that a particle-size gradient of the embedded functional particles results across the thickness of the nonwoven aggregate, at least in some areas or sections. Movement or displacement of the functional particles can be largely avoided with the nonwoven aggregate according to the invention. The nonwoven aggregate is also characterized by the fact that the properties of the layers or laminae are very precise and can be set or adjusted according to requirements. The nonwoven aggregate thus ensures a functionally reliable fixation and the realization of a particle-size gradient and can nevertheless be made with relatively little effort and at relatively low cost. The functional particles are protected against falling out of the nonwoven aggregate as well as against displacement or movement within the nonwoven aggregate in a very functionally reliable manner. A realized particle-size gradient is thus largely maintained in the nonwoven aggregate according to the invention. Compared to the known measures, these properties can be realized in a very functionally reliable and less complex manner. In addition, the nonwoven aggregate according to the invention allows a very precise gradation of the properties of the layers, for example with regard to the average pore size.

The invention is described in the following in more detail with reference to a drawing showing only one embodiment. Therein:

FIG. 1 is a sectional view of a nonwoven aggregate according to the invention in a first embodiment,

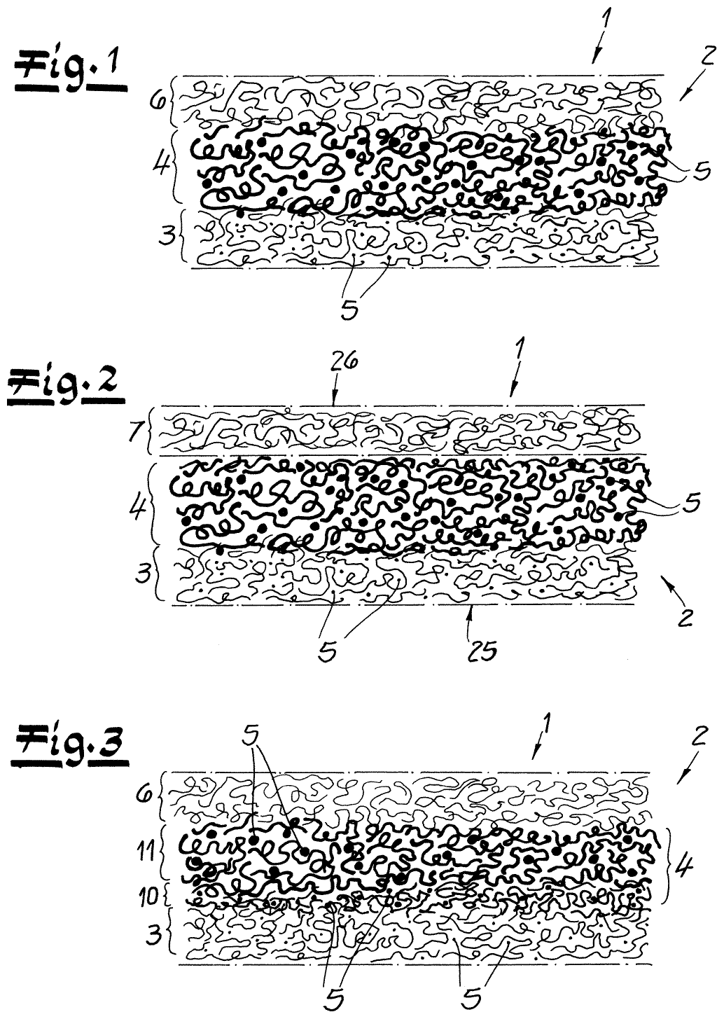

FIG. 2 is a sectional view of a nonwoven aggregate according to the invention in a second embodiment,

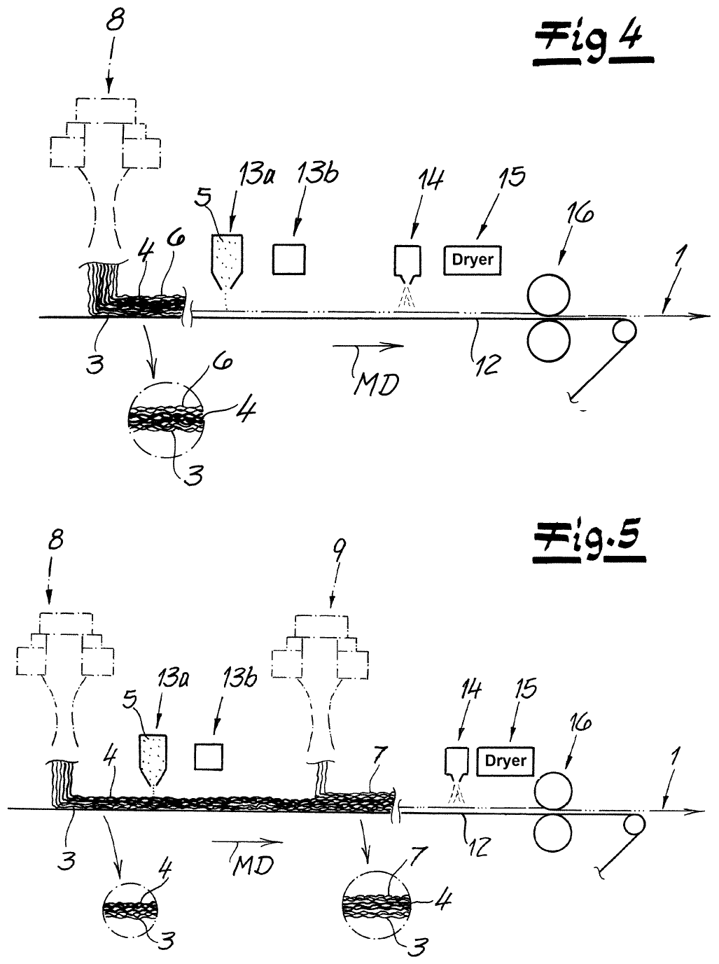

FIG. 3 is a sectional view of a nonwoven aggregate according to the invention in a third embodiment,

FIG. 4: a vertical section through an apparatus according to the invention for making a nonwoven aggregate according to the invention,

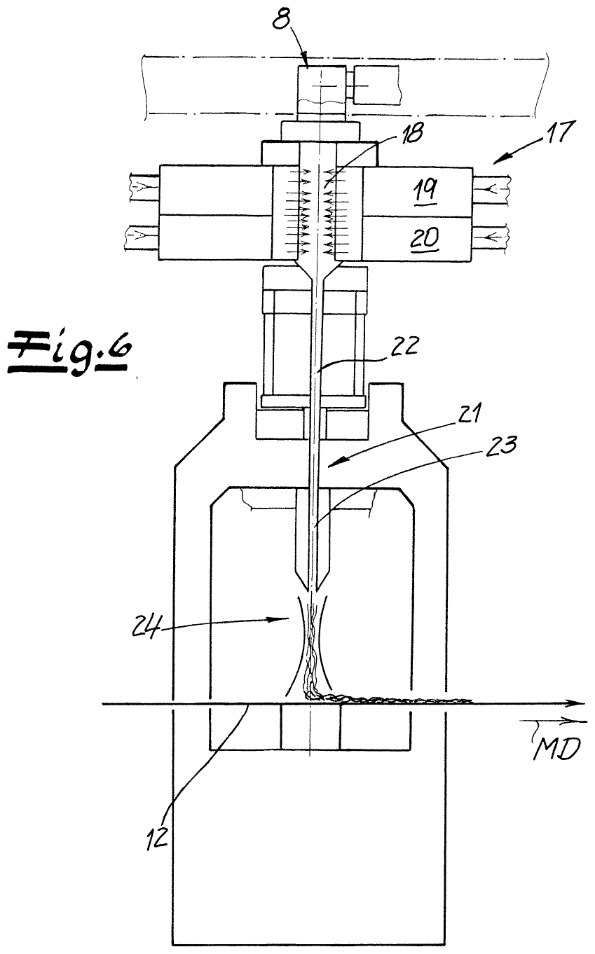

FIG. 5 is a vertical section through an apparatus according to the invention for making a nonwoven aggregate according to the invention in a second embodiment,

FIG. 6 is a vertical section through a part of the apparatus according to the invention for making a layer or spunbond layer.

FIG. 1 shows a nonwoven aggregate 1 according to the invention with a nonwoven lamina 2 with at least a first layer 3 and at least a second layer 4. Preferably and here, the first layer 3 and the second layer 4 are a spunbond nonwoven layer of continuous filaments. The nonwoven aggregate 1 according to FIG. 1 also preferably and here has a third layer 6 applied directly to the second layer 4 in the form of a spunbond nonwoven layer of continuous filaments. The second layer 4 has functional particles 5 embedded in the pores between the filaments of the second layer 4. The average pore size of the pores formed in the first layer 3 is smaller than the average pore size of the pores between the filaments of the second layer 4. Conveniently and here, functional particles 5 are also embedded in the pores between the filaments of the first layer 3 whose average size is preferably and here smaller than the average size of the functional particles 5 embedded in the pores of the second layer 4. Preferably and here, the functional particles 5 are embedded in the pores of the second layer 4 without adhesives or binders. Moreover and according to a preferred embodiment, this also applies to the functional particles 5 that are embedded in the pores of the first layer 3. The functional particles 5 may be absorption particles, in particular for absorbing fluid media.

The nonwoven lamina 2 or the first layer 3 and the second layer 4 and the third layer 6 are particularly preferably and here according to FIG. 1 spun from only one spinning beam or from only one spinneret. Preferably and here according to FIG. 1, the filaments of the first layer 3 and the filaments of the third layer 6 have a lower mean titer than the filaments of the second layer 4. Further preferably and here, the filaments of the third layer 6 have a higher mean titer than the filaments of the first layer 3. Here according to FIG. 1, the titer of the filaments of the first layer 3 may be about 2.0 den, the mean titer of the filaments of the second layer 4 may be about 6.0 den and the mean titer of the filaments of the third layer 6 may be about 2.5 den. The filaments of the first layer 3 and the second layer 4 and the third layer 6 are preferably and here crimped bicomponent filaments, the bicomponent filaments having a low-melting-point binder component and a high-melting-point second component. Here according to the figures, the low-melting-point binder may consist of or essentially consist of polyethylene and the second component or the high-melting-point component may consist of or essentially consist of polypropylene.

Preferably and here, the degree of crimp of the filaments of the first layer 3 and the filaments of the third layer 6 is lower than the degree of crimp of the filaments of the second layer 4.

According to a preferred embodiment and here according to the figures, the mass fraction of the first layer 3 is 5 to 60 wt % and the mass fraction of the second layer 4 is 30 to 85 wt % and the mass fraction of the third layer 6 IS 5 to 15 wt % relative to the aggregate of first layer 3, second layer 4 and third layer 6. The mass fractions preferably and here relate to the aggregate without functional particles or before the application and incorporation of the functional particles. Here according to the figures, the mass fraction of the first layer 3 may be 20 wt %, the mass fraction of the second layer 4 may be 70 wt % and the mass fraction of the third layer 6 may be 10 wt %, based on the aggregate of first layer 3, second layer 4 and third layer 6.

FIG. 2 shows a further embodiment of the nonwoven aggregate according to the invention. In the embodiment shown in FIG. 2, a nonwoven second lamina 7 of continuous filaments is present instead of a third nonwoven layer of continuous filaments. With regard to the layers or laminae, essentially the same description applies as for FIG. 1. The nonwoven second lamina 7 is spun from a further or second spinning beam 9 or a further or second spinneret and deposited on the second layer 4. Preferably and here, the nonwoven second lamina 7 is a spunbond. Preferably and here, the first layer 3 forms a first nonwoven aggregate outer face 25 or lower face and the nonwoven second lamina 7 preferably forms a second nonwoven aggregate outer face 26 or upper face opposite the first nonwoven aggregate outer face.

FIG. 3 shows a further embodiment of a nonwoven aggregate 1 according to the invention. In the embodiment according to FIG. 3, the nonwoven aggregate 1 has, analogous to the embodiment according to FIG. 1, a first layer 3 in the form of a nonwoven layer of continuous filaments and a third layer 6 in the form of a nonwoven layer of continuous filaments, the first layer 3 and the second layer 4 and the third layer 6 being spun from the same spinning beam 8 or from the same spinneret. The first layer 3 and the second layer 4 and the third layer 6 are preferably and here spunbond nonwoven layers. In the embodiment according to FIG. 3 and preferably, the second layer 4 consists of two partial layers 10, 11, the first partial layer 10 being adjacent the first layer 3 and the second partial layer 11 adjacent the third layer 6. In this way, the properties of the second layer 4 can be particularly finely adjusted or graded. In the embodiment according to FIG. 3 with the two partial layers 10, 11 of the second layer 4, the filaments of the first partial layer 10 of the second layer 4 preferably have a lower mean titer than the filaments of the second partial layer 11 of the second layer 4. Furthermore, preferably and here, the mean pore size of pores formed between the filaments of the first partial layer 10 is smaller than the mean pore size of pores formed between the filaments of the second partial layer 11. However, the mean pore size of pores formed between the first partial layer 10 is preferably and here larger than the mean pore size of pores formed between the filaments of the first layer 3 and the third layer 6. FIG. 3 also shows that a particle-size gradient results within the second layer 4 with regard to the average size of the embedded functional particles 5. Conveniently and here, the average particle size of the embedded particles decreases from the second partial layer 11 toward the first partial layer 10 and further toward the first layer 3. The mass fraction of the first partial layer 10 is preferably 15 to 30 wt % based on the aggregate of first layer 3, second layer 4 and third layer 6 and may be about 20 wt % here. The mass fraction of the second partial layer 11 is preferably 40 to 70 wt % relative to the aggregate of first layer 3, second layer 4 and third layer 6 and may be about 50 wt % here.

FIG. 4 shows an apparatus according to the invention for making a nonwoven aggregate 1 according to the invention in a first embodiment. The apparatus has a spinning beam 8 for spinning continuous filaments, as well as a receiving surface 12 is a mesh deposition belt for deposition of the filaments to form a nonwoven web. Preferably and here, filaments for a first layer 3 in the form of a nonwoven layer of continuous filaments can be made with the spinning beam 8 and deposited on the receiving surface 12. Preferably and here, the same spinning beam 8 can be used to produce filaments for a second layer 4 in the form of a nonwoven layer of continuous filaments, which filaments can be deposited on the first layer 3. Preferably and here, the same spinning beam 8 can be used to produce filaments for a third layer 6 in the form of a nonwoven layer of continuous filaments, which can be deposited on the second layer. The spinning beam 8 or the spinneret openings of the spinning beam 8 are preferably and here set up such that the filaments of the first layer 3 have a lower mean titer than the filaments of the second layer 4 and that the filaments of the third layer 6 have a higher mean titer than the filaments of the first layer 3, but a lower mean titer than the filaments of the second layer 4. Further preferably and here, the spinning beam 8 or the spinneret openings of the spinning beam 8 are provided such that the filaments of the first layer 3 and the second layer 4 and the third layer 6 are crimped multicomponent filaments and that the filaments of the second layer 4 have a higher degree of crimp than the filaments of the first layer 3 and the filaments of the third layer 6.

Preferably and here, the apparatus has a unit 13a, 13b for applying and injecting functional particles 5 onto and into the aggregate, the unit being downstream of the spinning beam 8 in the machine direction (MD direction). Preferably and here, the applying and injecting unit 13a, 13b has an applicator 13a and an injector 13b. The applying and injecting device 13a, 13b can preferably apply the functional particles to the aggregate and introduce them into the aggregate. The applicator 13a is preferably and here a spreader funnel. The injector 13b can be a device for generating acoustic pressure, for example by sound. Preferably and here, the apparatus according to the invention also has an applicator 14, in particular a spray nozzle, for applying a hydrophilizing agent, in particular a liquid hydrophilic lubricant, to the nonwoven aggregate. The applicator 14 is expediently connected downstream of the spinning beam 8 in the machine direction and downstream of the unit 13a, 13b for applying and injecting the functional particles 5. Further preferably and here, a dryer 15 for drying the applied or sprayed-on hydrophilic lubricant is provided downstream of the applicator 14 in the machine direction. The apparatus according to the invention here is suitably equipped with a device 16 for consolidating the nonwoven aggregate. This consolidator 16 can be a through-flow oven and/or a hot calender. It is advisable to feed the aggregate preconsolidated in the oven directly to the hot calender without significant cooling in order to utilize the product waste heat for effective hot calendering.

FIG. 5 shows a second embodiment of the apparatus according to the invention. For this second embodiment, essentially the same description applies as for FIG. 4. However, the apparatus according to the second embodiment has a second spinning beam 9 for making continuous filaments connected downstream of the first spinning beam 8 in the machine direction and which is upstream of the applicator 14 in the machine direction for applying a hydrophilic lubricant to the nonwoven aggregate. With the apparatus shown in FIG. 5, a first layer 3 in the form of a nonwoven layer of continuous filaments and a second layer 4 in the form of a nonwoven layer of continuous filaments can be made with the first spinning beam 8. A nonwoven second lamina 7 of continuous filaments can be deposited on the aggregate of the first layer 3 and the second layer 4 with the apparatus shown in FIG. 5 spun with the second spinning beam 9. The subsequent application of the hydrophilizing agent, in particular the liquid hydrophilic lubricant, by the applicator 14 and the drying of the hydrophilic lubricant by the dryer 15 is carried out analogously to the embodiment according to FIG. 4. This also applies to the consolidating of the nonwoven aggregate with the consolidator 16. In the apparatus according to FIG. 5 and preferably an applying and injecting unit 13a, 13b for applying and injecting functional particles 5 onto the aggregate is present assigned to the first layer 3 and the second layer 4 that is downstream of the spinning beam 8 in the machine direction and upstream of the spinning beam 9 in the machine direction. The applying and injecting unit 13a, 13b is thus preferably and here between the two spinning beams 8, 9. Preferably and here, the applying and injecting unit 13a, 13b has an applicator 13a a spreading funnel and an injector 13b preferably and here a device for generating acoustic pressure.

FIG. 6 shows the basic structure of a part of the apparatus according to the invention for making a layer or spunbond layer for the nonwoven aggregate according to the invention using the spunbond method, comprising a spinneret or the spinning beam 8 for spinning the continuous filaments for a nonwoven layer of continuous filaments. The continuous filaments spun by the spinneret or the spinning beam 8 are introduced into a cooler 17 with a cooling chamber 18. Preferably and here, air supply cabins 19, 20 are one above the other on two opposite sides of the cooling chamber 18. Air of different temperatures is preferably introduced into the cooling chamber 18 from the air supply cabins 19, 20 one above the other.

In a recommended embodiment, a stretcher 21 for stretching the continuous filaments is connected downstream of the cooler 3 in the direction of filament flow. Expediently and here, the stretcher 21 has an intermediate passage 22 that connects the cooler 17 to a stretching shaft 23 of the stretcher 21. Preferably and here, the unit comprising the cooler 17, the intermediate passage 22 and the stretching shaft 23 is a closed unit and apart from the supply of cooling air in the cooler 17, there is no other external air supply to this unit.

Conveniently and here, a diffuser 24 follows the stretcher 21 in the filament flow direction, through which the continuous filaments are guided. After passing through the diffuser 24, the continuous filaments are preferably and here deposited on a receiving surface 12 a mesh deposition belt. The mesh deposition belt is preferably and here an endlessly circulating mesh deposition belt. It is within the scope of the invention that the mesh deposition belt is permeable to air, so that process air can be extracted from below through the mesh deposition belt.

1. A nonwoven aggregate with at least one nonwoven lamina with at least one first layer and at least one second layer, the second layer being nonwoven and made of continuous filaments, wherein
the average pore size of pores formed in the first layer is smaller than the average pore size of the pores between the filaments of the second layer,
the second layer comprises functional particles embedded in pores between the filaments of the second layer, and
the first layer forms a barrier against the penetration of particles or functional particles with a size greater than 150 μm.

2. The nonwoven aggregate according to claim 1, wherein
at least one nonwoven third layer in the form of a nonwoven layer of continuous filaments is applied to the second layer or at least one nonwoven second lamina of continuous filaments is applied to the second layer,
the third layer or the nonwoven second lamina has an average pore size of the pores between the filaments that is smaller than the average pore size of the pores between the filaments of the second layer, and
the third layer or the nonwoven second lamina forms a barrier against the penetration of particles or functional particles with a size greater than 150 μm.

3. The nonwoven aggregate according to claim 1, wherein the functional particles are embedded in the pores of the second layer without adhesives or binders.

4. The nonwoven aggregate according to claim 1, wherein the functional particles are at least in part absorption particles for absorption of fluid media and/or at least in part adsorption particles.

5. The nonwoven aggregate according to claim 1, wherein the first layer is a nonwoven layer of continuous filaments and the first layer and/or the second layer and/or the third layer is a spunbond nonwoven layer and/or the nonwoven second lamina is a spunbond nonwoven lamina.

6. The nonwoven aggregate according to claim 1, wherein the first nonwoven lamina or the first layer and the second layer are spun from only one spinning beam or from only one spinneret.

7. The nonwoven aggregate according to claim 6, wherein the third layer is spun from the same spinning beam or from the same spinneret as the first and the second layers or the nonwoven second lamina is spun from a further or second spinning beam or from a further or second spinneret.

8. The nonwoven aggregate according to claim 5, wherein the filaments of the first layer and/or the filaments of the third layer or the nonwoven second lamina have a lower average titer than the filaments of the second layer and preferably the filaments of the third layer or the nonwoven second lamina have a higher average titer than the filaments of the first layer.

9. The nonwoven aggregate according to claim 1, wherein the filaments of the first layer and/or the filaments of the second layer and/or the filaments of the third layer or of the nonwoven second lamina are multicomponent filaments having at least one low-melting-point binder.

10. The nonwoven aggregate according to claim 5, wherein the degree of crimp of the filaments of the first layer and/or the filaments of the third layer or second nonwoven lamina is lower than the degree of crimp of the filaments of the second layer.

11. The nonwoven aggregate according to claim 9, wherein the proportion of the low-melting-point binder in the multicomponent filaments of the first layer and/or the third layer or of the nonwoven second lamina is higher than the proportion of the low-melting-point binder component in the multicomponent filaments of the second layer.

12. The nonwoven aggregate according to claim 9, wherein the binder proportion of the multicomponent filaments of the second layer is dimensioned such that in the event of swelling of the functional particles at least partial breaking of bonds between the filaments of the second layer is possible and for this purpose the binder proportion of the multicomponent filaments is 5 to 55 wt % based on the components of the multicomponent filaments.

13. The nonwoven aggregate according to claim 9, wherein the multicomponent filaments of the first layer and/or of the third layer or nonwoven second lamina are multicomponent filaments with.

14. The nonwoven aggregate according to claim 2 wherein the mass fraction of the first layer is 5 to 60 wt %, the mass fraction of the second layer is 30 to 85 wt %, and the mass fraction of the third layer or the nonwoven second lamina is 4 to 15 wt % based on the aggregate of first layer, second layer and third layer or nonwoven second lamina.

15. The nonwoven aggregate according to claim 1, wherein the second layer comprises at least two, preferably two partial layers, the filaments of the first partial layer of the second layer have a lower average titer than the filaments of the second partial layer of the second layer or the nonwoven second lamina and/or both partial layers contain multicomponent filaments, the multicomponent filaments of the first partial layer have a higher binder content than the multicomponent filaments of the second partial layer.

16. A method of making a nonwoven aggregate, comprising the steps of:
depositing at least a first layer on a mesh deposition belt;
depositing on the belt a second layer in the form of a nonwoven layer of continuous filaments by spinning out the continuous filaments from a spinning beam and depositing it the spun filaments on the first layer;
applying functional particles to the aggregate and embedded the particles between the filaments of the second layer, such that the first layer forms a barrier against penetration by particles or functional particles with a size greater than 150 μm.

17. The method according to claim 16, wherein the first layer is also made as a nonwoven layer of continuous filaments and the filaments for the first layer and the filaments for the second layer are spun with the same spinning beam.

18. The method according to claim 16, further comprising the step of:
making and depositing at least a third layer in the form of a nonwoven layer of continuous filaments or at least a nonwoven second lamina of continuous filaments on the second layer.

19. The method according to claim 16, further comprising the steps of:
applying the functional particles to the third layer or the nonwoven second lamina;
making the third layer or the nonwoven second lamina such that the functional particles penetrate the third layer or the nonwoven second lamina at least before solidification and/or compaction of the third layer or nonwoven second lamina and are embedded at least for the most part between the filaments of the second layer.

20. An apparatus for making a nonwoven aggregate, the apparatus comprising:
a spinning beam for spinning out continuous filaments and and depositing the filaments on a mesh deposition belt to form a nonwoven web, the spinning beam being used to produce filaments for at least one first layer or first nonwoven layer and for their deposition on the receiving surface, and for producing filaments for at least one second layer or second nonwoven layer and depositing the second layer on the first layer or first nonwoven layer, the spinning beam or the spinneret openings of the spinning beam being set up such that the filaments of the first layer have a lower mean titer than the filaments of the second layer and/or
the filaments of the first and second layers are crimped multicomponent filaments and the filaments of the second layer have a higher degree of crimp than the filaments of the first layer and/or
the filaments of the first and second layers are multicomponent filaments with a low-melting-point binder component and the binder component content of the filaments of the first layer is higher than the binder component content of the filaments of the second layer, and
at least one applying and/or injecting device is downstream of the spinning beam in the machine direction for applying and/or
injecting functional particles onto or into the aggregate.
